(19) United States
(12) Patent Application Publication
(10) Pub. No.: US 2026/0309690 A1
(43) Pub. Date: Oct. 8, 2026

(54) ARTIFICIAL ROOTS FOR SOIL NUTRIENT UPTAKE MONITORING

(71) Applicant: Trustees of Tufts College, Medford, MA (US)

(72) Inventor: Sameer Sonkusale, Lincoln, MA (US)

(73) Assignee: Trustees of Tufts College, Medford, MA (US)

(21) Appl. No.: 19/478,970

(22) PCT Filed: May 2, 2024
(86) PCT No.: PCT/US2024/027415

§ 371 Date: Oct. 27, 2025

Related U.S. Application Data
(60) Provisional application No. 63/463,684, filed on May 3, 2023.

(51) Int. Cl. G01N 33/24

(52) U.S. Cl. CPC G01N 33/245

(57) ABSTRACT

A biomimetic plant for implantation into soil includes an ion-sensitive electrode, a biomimetic root, and readout-and-communication circuitry. The biomimetic root uses energy in its environment to cause water to flow continuously past the ion-sensitive electrode. The readout-and-communication circuitry transmits data that results from measurement by the ion-sensitive electrode to a central base station.

### RELATED APPLICATIONS

This application claims the benefit of the May 3, 2023 priority date of U.S. Provisional Application 63/463,684, the contents of which are incorporated herein by reference.

### STATEMENT OF GOVERNMENT RIGHTS

This invention was made with government support under grant 1935555 awarded by the National Science Foundation. The government has certain rights in the invention.

### BACKGROUND

It is often useful to measure concentrations of various substances in soil. A known way to do so is to dig up a soil sample and bring it to a laboratory for analysis.

A disadvantage of this method is that it does not provide continuous measurements. As a result, it is difficult to ascertain the dynamic behavior of the various substances in the soil.

### SUMMARY

It is important to monitor nutrient uptake in plants to optimize fertilizer treatment and reduce fertilizer run off. Current methods to monitor nutrient uptake rely on measurement in soil. This fails to capture the true bioavailability of nutrients in plants.

In one aspect, the invention features a soil-monitoring platform in the form of a biomimetic plant for implantation into the soil. The biomimetic plant comprises an ion-sensitive electrode, a biomimetic root, a biomimetic leaf, and readout-and-communication circuitry. The biomimetic root uses energy in an environment thereof to cause soil water to flow continuously past the ion-sensitive electrode. The readout-and-communication circuitry measures and transmits data that results from measurement by the ion-sensitive electrode to a central base station.

Embodiments include those in which the root is configured to cause water to flow by causing transpiration of water from the soil using a biomimetic leaf. This allows for continuous fluid flow.

Further embodiments include those in which the root comprises threads that wick water from the soil and the biomimetic leaf comprises an absorbent pad exposed to air for enabling water drawn by the threads to evaporate. Among these are embodiments that include a temperature-control device to control both evaporation rate from the pad and flow rate through the biomimetic root. Examples include a heater, which increases flow rate and evaporation rate when activated, and a cooler that decreases flow rate and evaporation rate when activated.

In some embodiments, the biomimetic plant is configured to simulate the properties of a target plant that is being cultivated. The biomimetic plant, in this case, effectively becomes a digital twin, or in-silico twin, of this cultivated target plant. In either case, the result is a realistic sensor model that can provide a true measure of nutrient uptake for the target plant. A suitable model is a hybrid in-silico model in which the biomimetic plant provide a source of measurements to be used by a data-processing system to estimate nutrient uptake by the target plant and to predict nutrient requirements based on that estimate. Such a model is hybrid because it includes an in-silico component, namely the data-processing system, and a non-in-silico component, namely the biomimetic plant itself.

A hybrid in-silico model of this type comprises capillary-driven passive microfluidic channels that function as an artificial root, electrochemical sensors, and computational intelligence embodied in the data-processing system.

Natural capillary-driven transport in threads provides continuous sampling of soil water that is driven by evaporation of the water itself. This effectively mimics the combined functions of the target plant's roots and its xylem. The uptake of water from the soil measured by this hybrid in-silico model tracks uptake in plants. Like that in plants, it depends on the soil's saturation, temperature, and humidity. The hybrid in-silico model therefore serves as a digital twin of a target plant.

In some embodiments, the biomimetic plant comprises screen-printed electrochemical sensors along a microfluidic channel formed by a thread. Such sensors provide real-time measurement of soil nutrients.

Communicating the relevant data requires electronic circuitry for real-time analysis and transmission. In some embodiments, a power source to carry this out includes aa metal-air battery, such as a zinc-air battery to power low-power electronic circuitry.

Still other embodiments include those in which the biomimetic plant is configured to function as an in-silico twin of a target plant. Among these are embodiments in which the biomimetic plant transports water upward from the soil at a rate that is proportional to a rate at which a target plant transports water upwards from said soil and those in which the biomimetic plant is configured such that flow of fluid through the biomimetic plant mimics nutrient uptake by a target plant.

In some embodiments, the apparatus includes a monitor at the central base station. This monitor receives the measurements remotely via a network and uses those measurements for inferring a state of a target plant. Among these are embodiments that include construction of a digital twin of a target plant, with that digital twin having been trained to emulate a physiological process of the target plant based on those measurements. Also among the embodiments are those that include a machine-learning system that has been trained to emulate a physiological process of a target plant based on training data that comprises or is derived from measurements obtained from the biomimetic plant. Among these embodiments are those in which the machine-learning system has been trained to emulate the target plant's circadian rhythms, the flow velocity of the plant's sap, and the stage of plant growth. The integration of collected data with machine-learning technology provides accurate prediction and modeling of diurnal sap flow speed, growth stage, and other physiological parameters. An example of such a machine-learning system is a neural network that has been trained to emulate physiological processes of a target plant based on training data that has been derived from measurements obtained from said biomimetic plant.

Additional embodiments include those in which the biomimetic plant includes an acoustic transducer, those in which the biomimetic plant includes a transceiver that transmits a homing signal.

Still other embodiments include probes and electrodes disposed along the probes. Examples of electrodes include electrochemical electrodes, such as screen-printed electrochemical electrodes, ring electrodes, and ion-sensitive electrodes. These probes penetrate the soil and expose the electrodes to soil at different depths below the ground. Among these are embodiments having first and second probes that extend vertically by different extents into the soil. In such embodiments, the first probe comprises first electrodes that sample from a first subsurface layer and second probe comprises second electrodes that sample from a second subsurface layer that extends below the first subsurface layer.

In still other embodiments, the biomimetic plant is but one of many biomimetic plants that are constituents of the soil-monitoring platform. This permits sampling of soil over a broad area and averaging of results as well as providing information about the spatial distribution and extent of various soil properties measured by the biomimetic plant.

In addition to providing real-time measurements of actual nutrients in the soil and in the plant, the biomimetic plant is also useful for providing data to be used for predicting plant behavior under different environmental conditions, for enhancing understanding of plant responses in specific temporal and spatial contexts, and for planning interventions, such as fertilizer treatment. By applying fertilizer only when necessary, the biomimetic plant plays a role in reducing fertilizer runoff.

In another aspect, the invention features a method that includes receiving a signal from a biomimetic plant. This signal comprises information concerning flow of water along an artificial root of the biomimetic plant. The signal being derived from an ion-sensitive electrode on the biomimetic plant.

Among the practices of the method include those in which receiving the signal comprises receiving a signal from a first electrode at a first time, receiving a signal from a second electrode at a second time that is later than the first time, the second electrode being disposed above the first electrode, determining a different between the first time and the second time, and, based on the difference, inferring a rate at which fluid climbs from the first electrode to the second electrode.

These and other features of the invention will be apparent from the following detailed description and its accompanying figures, in which:

### DESCRIPTION OF THE FIGURES

FIG. 1 shows a platform having sensor motes,

FIG. 2 shows one of the sensor motes shown in FIG. 1,

FIG. 3 shows details of the biomimetic root shown in FIG. 2,

FIG. 4 shows an alternative embodiment of the sensor mote shown in FIG. 2. and

FIG. 5 shows a data-processing system in communication with the platform of FIG. 1.

### DETAILED DESCRIPTION

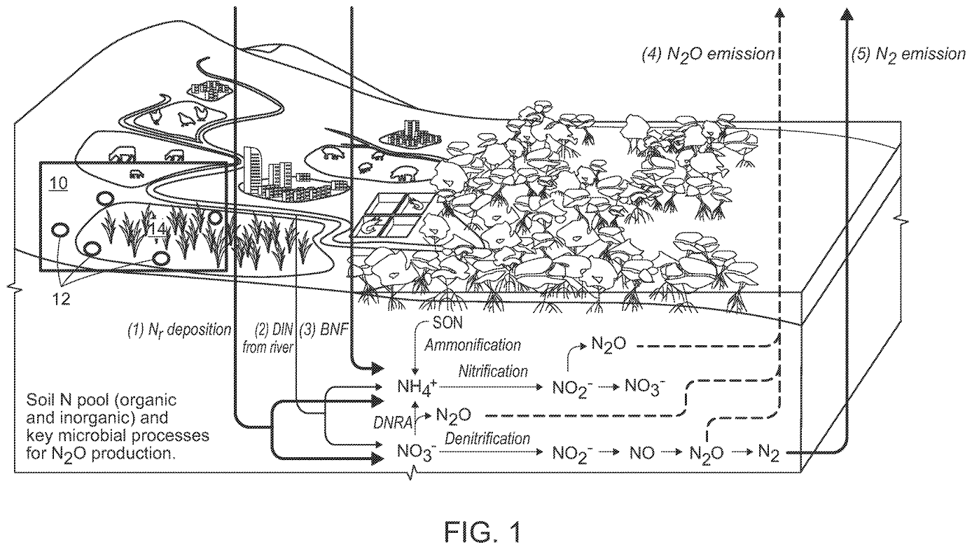

FIG. 1 shows a distributed soil monitoring platform 10 that comprises biomimetic plants 12 that have been spatially distributed within a region 14 whose soil is to be monitored. A typical soil-monitoring platform 10 uses multiple biomimetic plants 12 of the same type. Doing so improves sensitivity from statistical calibration. These biomimetic plants 12 carry out their function by extracting using energy present in the environment to carry out useful work.

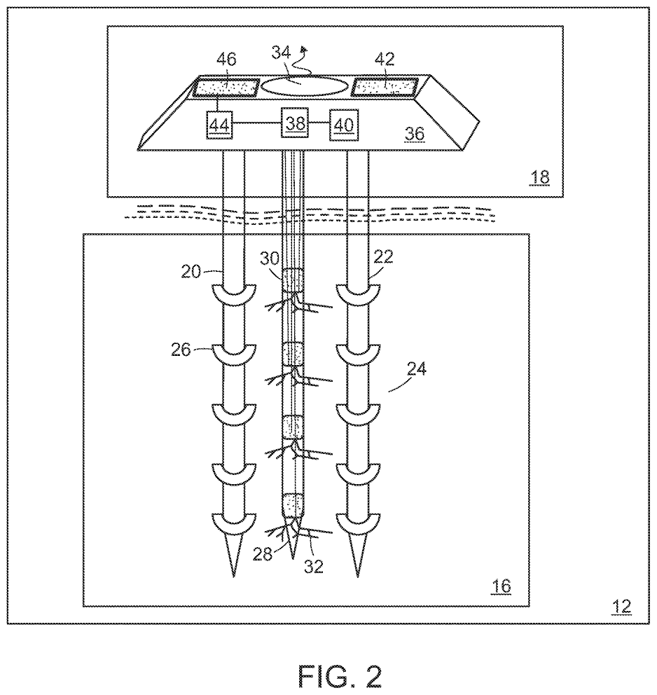

Referring now to FIG. 2, each biomimetic plant 12 comprises an underground portion 16 and an above-ground portion 18.

The underground portion 18 comprises first and second probes 20, 22, each of which is a soil-penetrable active probe. The first and second probes 20, 22 comprise electrode arrays 24. Each electrode array 24 comprises ring electrodes 26 at varying depths along the first or second probe 20, 22. These arrays 24 permit measurement of soil impedance between the first and second probes 20, 22. Such a measurement provides real-time indirect measurement of the soil's moisture content and its salinity.

In some embodiments, the first and second probes 20, 22 have different lengths. This allows electrode arrays 24 on different probes to make measurements within different ranges of depths. For example, if the second probe 22 extends more deeply than the first probe 20, placement of an electrode array 24 near the distal tip of the second probe 22 permits measurements from depths not accessible from an electrode array 24 on the first probe 20.

Each ring electrode 26 comprises a suitable conductive material. Because of its expected long-term exposure to soil, a useful choice of conductive material is an inert conductive material. Examples of such inert conductive materials include stainless steel, gold, carbon, particularly in the form of graphene, PEDOT: PSS, MXene, and any transition metal dichalcogenide.

The underground portion 18 further includes a biomimetic root 28 that harnesses potential energy present in the environment to move water past ion-sensitive electrodes disposed along the length of the root 28.

In one embodiment, the biometric root 28 features threads 32 that extend into the surrounding soil to draw water into a hollow interior of the root 27. An upper end of the root 28 couples to a biomimetic leaf in the form of an absorbent pad 34 that is exposed to the air. To control evaporation, some embodiments incorporate a temperature controller 35 in thermal communication with the pad 34. In some embodiments, the temperature controller 35 comprises a heater that increases evaporation rate. This is particularly useful for operation in high humidity or low temperature. In other embodiments, the temperature controller 35 includes a cooler in thermal communication with the pad 34 to decrease evaporation rate. A useful place to position the temperature controller 35 is on the surface of the pad 34, for example, on a lower or upper surface thereof. Embodiments include those in which the energy required by the temperature controller 35 comes from the thermoelectric effect.

Control over evaporation rate also provides an indirect way of controlling the rate at which fluid flows due to wicking. Maintaining a lower temperature slows down the wicking rate whereas maintaining a higher temperature increases the wicking rate. Control over the wicking rate is useful for sensing concentrations of soil nutrients.

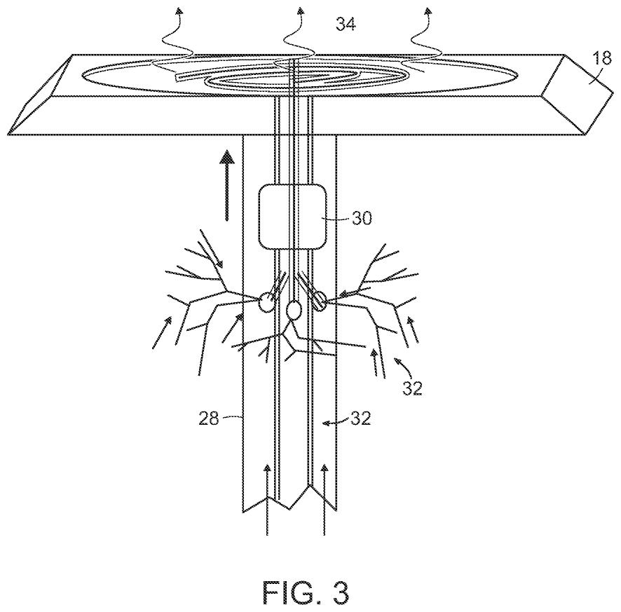

Referring now to FIG. 3, a suitable thread 32 is a cellulosic thread that readily absorbs water from the soil and transports that water upward, thereby mimicking the action of an actual plant's xylem. In some embodiments, the thread 32 is a multi-filament textile thread that has been rendered hydrophilic through chemical or plasma treatment thereof. When exposed to the soil, these threads 32 wick the soil moisture naturally filtering the sediment and carrying dissolved micronutrients with it. To increase surface area for water intake, it is useful to arrange the threads 32 as shown to form something that mimics a typical plant's root system. Natural capillary action along the length of the thread 32 carries water from moist portions of the thread upwards towards drier portions of the threads 32.

In this embodiment, the ion-sensitive electrodes 30 are embedded in or otherwise in fluid communication with the threads 32. In a typical embodiment, the ion-sensitive electrodes 30 comprise microwires placed in contact with the threads 32. These ion-sensitive electrodes 30 provide signals indicative of ion concentrations in the water that is being transported by the threads 32. The particular ions depend on the nature of the ion-sensitive electrodes 30. Examples of ion-sensitive electrodes 30 include those that are sensitive to potassium ions, nitrate ions, and ammonium ions. Other examples include those that are sensitive to chloride ions and/or to ions of calcium, magnesium, sodium, boron, chloride zinc, iron, molybdenum, nickel, copper, and manganese.

Since the electrodes 30 are a known distance apart, it is possible, with the addition of a clock signal, to measure flow rate. This flow rate would correspond to the time delay required to achieve the same measurement value at two successive electrodes 30. Using information about which electrodes 30 are sensitive to which ionic species provides a basis for estimating flow rate of different ionic species through the xylem of the plant whose behavior is being simulated.

Some embodiments further include a mechanism for carrying out ion-exchange desalination. This promotes selectivity to ammonium ions by removing cationic interference.

As a result of capillary action, the threads 32 wick water from the soil. As a result of capillary action, this water climbs upwards to the pad 34, from which it evaporates. As the water climbs upwards, it comes into contact with the ion-sensitive electrodes 30 along the way.

Evaporation from the pad 34 sustains continuous capillary induced flow through the microfluidic threads 32 beyond the saturation capacity of the pad 34.

The mechanism that drives a constant flow of water up the root 28 towards the pad 34 uses a mechanism similar to transpiration of water through a plant's leaves. As a result the ion sensitive electrodes 30 continuously sample water without the need for external power, for example to drive a pump.

Most of the micronutrients contained in the soil enter the plants through its roots using osmosis and diffusional gradients. By measuring real-time concentrations of micronutrients through continuous sampling of the soil water, the biomimetic plant 12 captures the true bioavailability of micronutrients to the plant, and not just the absolute concentration found in the soil. Since the sampling takes place in a manner similar to that relied upon by an actual living plant, information from the biomimetic plant 12 is more likely to reflect the true bioavailability of micronutrients, and not merely what is carried by water in the soil.

The above-ground portion 18 includes readout-and-communication circuitry 36. This circuitry 36 comprises a microcontroller 38, a long-range wireless transceiver 40, and an acoustic transducer 42. To provide power to carry out these functions, the above-ground portion 18 includes a rechargeable battery 44 and a solar cell 46 that recharges the battery 44. However, alternative embodiments obtain power by harvesting electrochemical energy from the environment using natural electrolytes already present in the soil.

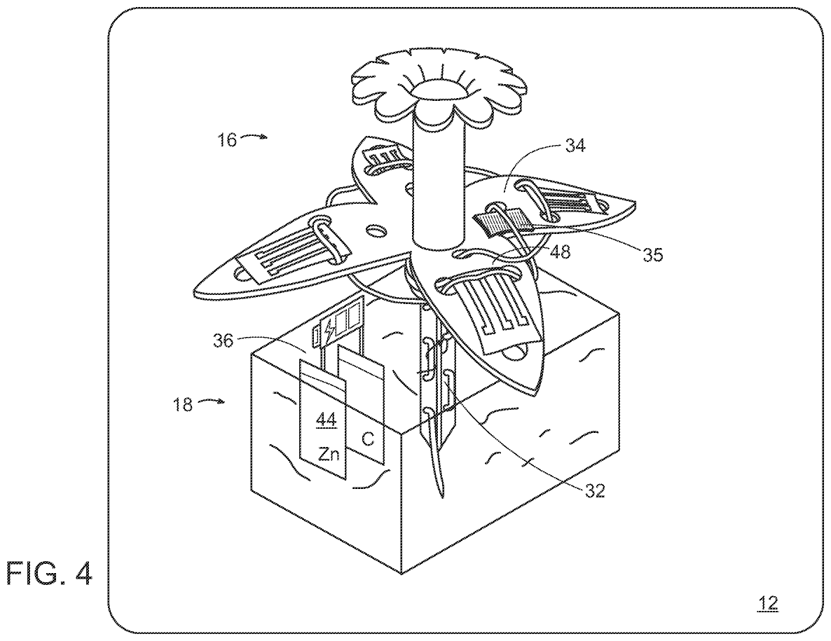

In an alternative embodiment, shown in FIG. 4, the battery 44 comprises a metal-air battery that generates an electrical potential as a result of metal oxidizing with air. A particularly useful battery 44 of this type is one in which the anode comprises zinc. In such embodiments, atmospheric oxygen interacts with the cathode to form hydroxyl ions which migrate into the zinc metal and form zincate, releasing electrons to travel to the cathode. The zincate ultimately decays into zinc oxide and water, the latter returning to the electrolyte. Water and hydroxyl from the anode are thus recycled at the cathode, ensuring that water is not consumed in the process. To provide an adequate steady voltage, it is also useful to include a boost converter.

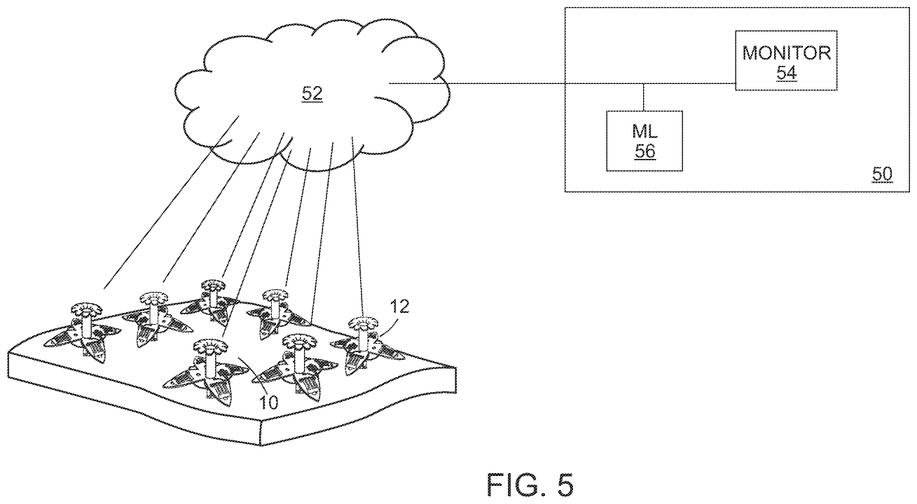

Referring now to FIG. 5, the readout-and-communication circuitry 36 records measurements made by the biomimetic plant 12, digitizes those measurements, and transmits the resulting data to a central base station 50 via a network 52. This base station 50 can be two or more kilometers away. The biomimetic plant 12, with its low-power requirements, is thus able to operate unattended for months or even years, all the while providing data to the base station 50.

The base station 50 includes a monitor 54 and a machine-learning system 56. A suitable machine-learning system 56 incorporates a neural network that has been trained on data from the ion-sensitive electrodes 30 using real data from a target plant as labels. This provides a way to train the machine-learning system 56 to function as a digital twin for the target plant.

In some cases, it may be necessary to locate a particular biomimetic plant 12. For example, it may have to be serviced. At the end of its lifetime, it will have to be removed. To facilitate being found, the biomimetic plant 12 emits an audible chirp through its acoustic transducer 42. In the alternative, the biomimetic plant 12 uses its transceiver 40 to transmit a suitable homing signal.

Further details can be found in the attached appendix, which is incorporated herein by reference.

Having described the invention and a preferred embodiment thereof, what is claimed as new and secured by letters patent is:

1. An apparatus comprising a soil-monitoring platform for providing continuous measurements of substances in soil, wherein said soil-monitoring platform comprises a biomimetic plant for implantation into said soil, wherein said biomimetic plant comprises an ion-sensitive electrode, a biomimetic root, a biomimetic leaf, and readout-and-communication circuitry, wherein said biomimetic root uses energy in an environment thereof to cause water to flow continuously past said ion-sensitive electrode and wherein said communication circuitry transmits data that results from measurement by said ion-sensitive electrode to a central base station.

2. The apparatus of claim 1, wherein said root is configured to cause water to flow by causing transpiration of water from said soil, wherein said root comprises threads that wick water from said soil, and wherein said biomimetic leaf comprises an absorbent pad exposed to air for enabling water drawn by said threads to evaporate.

3. (canceled)

4. The apparatus of claim 1, wherein said biomimetic leaf comprises a heater and an absorbent pad that is exposed to air and that is in fluid communication with said biomimetic root to enable drawn by said biomimetic root to evaporate from said absorbent pad, wherein activating said heater increases evaporation rate from said absorbent pad and increase flow rate through said biomimetic root.

5. The apparatus of claim 1, wherein biomimetic leaf comprises an absorbent pad exposed to air for enabling water drawn by said biomimetic root to evaporate from said absorbent pad and a cooler that, when activated to cool said absorbent pad, reduces evaporation rate from said absorbent pad and reduces flow rate through said biomimetic root.

6. The apparatus of claim 1, further comprising a zinc-air battery to provide power for said communication circuitry.

7. (canceled)

8. The apparatus of claim 1, wherein said biomimetic plant is configured to transport water upwards from soil at a rate that is proportional to a rate at which a target plant transports water upwards from said soil.

9. The apparatus of claim 1, wherein said biomimetic plant is configured such that flow of fluid through said biomimetic plant mimics nutrient uptake by a target plant.

10. The apparatus of claim 1, wherein said apparatus further comprises a monitor at said central base station, wherein said monitor receives said measurements remotely via a network, and wherein said monitor uses said measurements for inferring a state of a target plant.

11. The apparatus of claim 1, further comprising a digital twin of a target plant, wherein said digital twin has been trained to emulate a physiological process of said target plant.

12. The apparatus of claim 1, further comprising a machine-learning system that has been trained to emulate a physiological process of a target plant based on training data that comprises measurements obtained from said biomimetic plant.

13. The apparatus of claim 1, further comprising a digital twin of a target plant, wherein said digital twin has been trained to emulate circadian rhythm and growth stage of said target plant.

14. The apparatus of claim 1, further comprising a machine-learning system that has been trained to emulate circadian rhythm of a target plant based on training data that has been derived from measurements obtained from said biomimetic plant.

15. The apparatus of claim 1, further comprising a neural network that has been trained to emulate physiological processes of a target plant based on training data that has been derived from measurements obtained from said biomimetic plant.

16. (canceled)

17. The apparatus of claim 1, wherein said biomimetic plant further comprises a heater under said absorbent pad.

18. The apparatus of claim 1, wherein said biomimetic plant further comprises an acoustic transducer.

19. The apparatus of claim 1, wherein said biomimetic plant further comprises a transceiver that transmits a homing signal.

20. The apparatus of claim 1, wherein said biomimetic plant comprises an electrode array that comprises probes having ring electrodes disposed thereon.

21. (canceled)

22. The apparatus of claim 1, wherein said biomimetic root comprises first and second probes that extend vertically by different extents into said soil, wherein said first probe comprises first electrodes that sample from a first subsurface layer and second probe comprises second electrodes that sample from a second subsurface layer that extends below said first subsurface layer.

23. A method comprising receiving a signal from a biomimetic plant, wherein said signal comprises information concerning flow of water along an artificial root of said biomimetic plant, said signal being derived from an ion-sensitive electrode on said biomimetic plant.

24. The method of claim 23, wherein receiving said signal comprises receiving a signal from a first electrode at a first time, receiving a signal from a second electrode at a second time that is later than said first time, said second electrode being disposed above said first electrode, determining a different between said first time and said second time, and, based on said difference, inferring a rate at which fluid climbs from said first electrode to said second electrode.
